(19) United States
(12) Patent Application Publication
(10) Pub. No.: US 2018/0369458 A1
(43) Pub. Date: Dec. 27, 2018

(54) Irrigated Catheter with Retrograde Flow

(71) Applicant: St. Jude Medical, Cardiology Division, Inc., St. Paul, MN (US)

(72) Inventors: Jan O. Mangual-Soto, Rho (IT); Craig Markovitz, Leipzig (DE); Chunlan Jiang, N. Crystal, MN (US); Louis-Philippe Richer, Montreal (CA)

(21) Appl. No.: 16/017,449

(22) Filed: Jun. 25, 2018

Related U.S. Application Data
(60) Provisional application No. 62/525,028, filed on Jun. 26, 2017.

(51) Int. Cl. A61M 1/00; A61B 18/14

(52) U.S. Cl. CPC A61M 1/0058; A61B 2018/00363; A61M 1/0066; A61B 18/1492

(57) ABSTRACT

An irrigated ablation system with retrograde flow includes one or more medical devices (e.g., an ablation catheter and sheath) encompassing an irrigation lumen that terminates distally at an irrigation orifice and a drainage lumen that terminates distally at a drainage orifice. One or more pumps are coupled to the irrigation lumen and to the drainage lumen to deliver irrigant through the irrigation lumen and to extract fluid through the at least one drainage lumen. For example, a peristaltic pump can be used to simultaneously deliver irrigant through the irrigation lumen and to extract an equivalent volume of fluid through the drainage lumen. Alternatively, a feedback controller can be used to monitor parameters, such as impedance, pressure, ablation time, and/or irrigant volume, and control based thereon the rate at which the pump extracts fluid.

### CROSS-REFERENCE TO RELATED APPLICATIONS

This application claims the benefit of U.S. provisional application No. 62/525,028, filed 26 Jun. 2017, which is hereby incorporated by reference as though fully set forth herein.

### BACKGROUND

The instant disclosure relates to devices for use in medical procedures, such as cardiac ablation. In particular, the instant disclosure relates to an irrigated ablation system including controllable drainage to alleviate in situ fluid accumulation.

Catheters are used for an ever-growing number of procedures, such as diagnostic, therapeutic, and ablative procedures, to name just a few examples. Typically, the catheter is manipulated through the patient's vasculature and to the intended site, for example, a site within the patient's heart. The catheter typically carries one or more electrodes (in the case of so-called “electrophysiology catheters”) or other diagnostic or therapeutic devices, which can be used for ablation, diagnosis, cardiac mapping, or the like.

Irrigated electrophysiology catheters are also known. An irrigated electrophysiology catheter is an electrophysiology catheter that is equipped to deliver an irrigation fluid, such as saline, to a location proximate the electrodes. The irrigation fluid (or “irrigant”) serves, for example, to cool the electrodes or to disperse body fluids therefrom, to cool or bathe surrounding tissue, and/or to couple the electrodes to the tissue surface in the case of relatively highly conductive fluid(s).

In many irrigated electrophysiology catheters, a peristaltic pump is used to deliver the irrigation fluid. Typical peristaltic pumps operate by rotating a number of rollers mounted on a rotor to periodically compress an irrigation tube between the rollers and a pump housing or clamp, which forces the irrigation fluid through the irrigation tube.

An epicardial approach is used in the treatment of certain arrhythmias, such as ventricular tachycardia, by catheter ablation (e.g., radiofrequency ablation). During such procedures, irrigant can accumulate in the pericardial space, which can lead to inflammation (and patient discomfort), increased pericardial pressure, and anomalous catheter localization. The presence of a medical device in the pericardial sac can also lead to the buildup of bodily secretions, which can have similar effects.

Thus, it can be desirable to drain fluid, both irrigant and bodily secretions, from the pericardial sack during such procedures. Typically, drainage is done manually by the practitioner or an assistant.

### BRIEF SUMMARY

Disclosed herein is an irrigated ablation system including at least one medical device including at least one irrigation lumen extending along a length of the at least one medical device and having a distal terminus at an irrigation orifice that extends through a wall of a distal portion of the at least one medical device; and at least one drainage lumen extending along the length of the at least one medical device and having a distal terminus at a drainage orifice that extends through the wall of the distal portion of the at least one medical device. The irrigated ablation system further includes at least one pump coupled to the at least one irrigation lumen and to the at least one drainage lumen, wherein the at least one pump is operable to deliver irrigant through the at least one irrigation lumen and to extract fluid through the at least one drainage lumen.

In aspects of the disclosure, the at least one medical device includes an ablation catheter; and a sheath dimensioned to receive the ablation catheter therethrough. The ablation catheter can include the at least one irrigation lumen and the irrigation orifice, and the sheath can include the at least one drainage lumen and the drainage orifice.

It is contemplated that the rate at which the at least one pump extracts fluid through the at least one drainage lumen can equal a rate at which the at least one pump delivers irrigant through the at least one irrigation lumen. For example, the at least one pump can include a peristaltic pump simultaneously operable to deliver irrigant through the at least one irrigation lumen and to extract fluid through the at least one drainage lumen. For example, the peristaltic pump can include a rotor, wherein an interface tubing for the at least one irrigation lumen and an interface tubing for the at least one drainage lumen are positioned on opposing sides of the rotor.

In other embodiments, a rate at which the at least one pump extracts fluid through the at least one drainage lumen is independent of a rate at which the at least one pump delivers irrigant through the at least one irrigation lumen. For example, the rate at which the at least one pump extracts fluid through the at least one drainage lumen can be constant. Alternatively, a controller can be included that is operable to control the rate at which the at least one pump extracts fluid through the at least one drainage lumen responsive to one or more monitored parameters, which can be selected from the group consisting of impedance; pressure; ablation time; and irrigant volume.

Also disclosed herein is a method of performing cardiac ablation. The method includes introducing at least one medical device into a pericardial space, with the at least one medical device including: at least one irrigation lumen extending along a length of the at least one medical device and having a distal terminus at an irrigation orifice that extends through a wall of a distal portion of the at least one medical device and that is open to the pericardial space; and at least one drainage lumen extending along the length of the at least one medical device and having a distal terminus at a drainage orifice that extends through the wall of the distal portion of the at least one medical device and that is open to the pericardial space. The method also includes: coupling at least one pump to the at least one irrigation lumen and the at least one drainage lumen such that the at least one pump is operable to deliver irrigant through the at least one irrigation lumen and to extract fluid through the at least one drainage lumen; and ablating cardiac tissue using the at least one medical device while delivering irrigant through the at least one irrigation lumen and extracting fluid through the at least one drainage lumen.

According to aspects of the disclosure, the step of introducing at least one medical device into a pericardial space includes introducing an ablation catheter into the pericardial space via a sheath, wherein the ablation catheter comprises the at least one irrigation lumen and the irrigation orifice, and wherein the sheath comprises the at least one drainage lumen and the drainage orifice.

The rate at which the at least one pump extracts fluid through the at least one drainage lumen can equal a rate at which the at least one pump delivers irrigant through the at least one irrigation lumen. For example, the step of coupling at least one pump to the at least one irrigation lumen and the at least one drainage lumen such that the at least one pump is operable to deliver irrigant through the at least one irrigation lumen and to extract fluid through the at least one drainage lumen can include coupling a peristaltic pump to the at least one irrigation lumen and the at least one drainage lumen such that the peristaltic pump is simultaneously operable to deliver irrigant through the at least one irrigation lumen and to extract fluid through the at least one drainage lumen. By way of further explanation, the step of coupling a peristaltic pump to the at least one irrigation lumen and the at least one drainage lumen such that the peristaltic pump is simultaneously operable to deliver irrigant through the at least one irrigation lumen and to extract fluid through the at least one drainage lumen can include: positioning an interface tubing for the at least one irrigation lumen on a first side of a pump rotor; and positioning an interface tubing for the at least one drainage lumen on a second side of the pump rotor opposite the first side of the pump rotor.

In additional embodiments of the disclosure, the rate at which the at least one pump extracts fluid through the at least one drainage lumen can be independent of a rate at which the at least one pump delivers irrigant through the at least one irrigation lumen. For example, the rate at which the at least one pump extracts fluid through the at least one drainage lumen can be constant. As another example, the method can include: monitoring one or more parameters during the ablating step; and adjusting the rate at which the at least one pump extracts fluid through the at least one drainage lumen responsive to the one or more monitored parameters, which can be selected from the group consisting of impedance; pressure; ablation time; and irrigant volume.

The instant disclosure also provides an irrigated ablation system including: a medical device including: an irrigation lumen extending along a length of the medical device and having a distal terminus at an irrigation orifice that extends through a wall of a distal portion of the medical device; and a drainage lumen extending along the length of the medical device and having a distal terminus at a drainage orifice that extends through the wall of the distal portion of the medical device; at least one pump coupled to the irrigation lumen and to the at least one drainage lumen, wherein the at least one pump is operable to deliver irrigant through the irrigation lumen and to extract fluid through the drainage lumen; and a control to toggle the at least one pump between a first operating mode, wherein a rate at which the at least one pump extracts fluid through the drainage lumen equals a rate at which the at least one pump delivers irrigant through the irrigation lumen, and a second operating mode, wherein the rate at which the at least one pump extracts fluid through the drainage lumen is independent of the rate at which the at least one pump delivers irrigant through the irrigation lumen.

In aspects of the disclosure, the second operating mode includes a first sub-mode, wherein the rate at which the at least one pump extracts fluid through the drainage lumen is constant, and a second sub-mode, wherein the rate at which the at least one pump extracts fluid through the drainage lumen is responsive to one or more monitored parameters selected from the group consisting of impedance; pressure; ablation time; and irrigant volume.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic depiction of an electrophysiology catheter according to aspects of the instant disclosure.

FIG. 2 is a schematic depiction of a peristaltic pump, such as may be employed in connection with the present teachings.

FIG. 3 illustrates another embodiment of the instant disclosure, including an electrophysiology catheter and an introducer sheath.

### DETAILED DESCRIPTION

For the sake of illustration, certain embodiments of the disclosure will be explained herein with reference to an irrigated radiofrequency (“RF”) ablation catheter, such as the FlexAbility™ irrigated ablation catheter of Abbott Laboratories. It should be understood, however, that the present teachings may be applied to good advantage in other contexts as well. Further, insofar as the construction of irrigated radiofrequency ablation catheters will be familiar to those of ordinary skill in the art, the details thereof will only be described herein to the extent necessary to understand the instant disclosure.

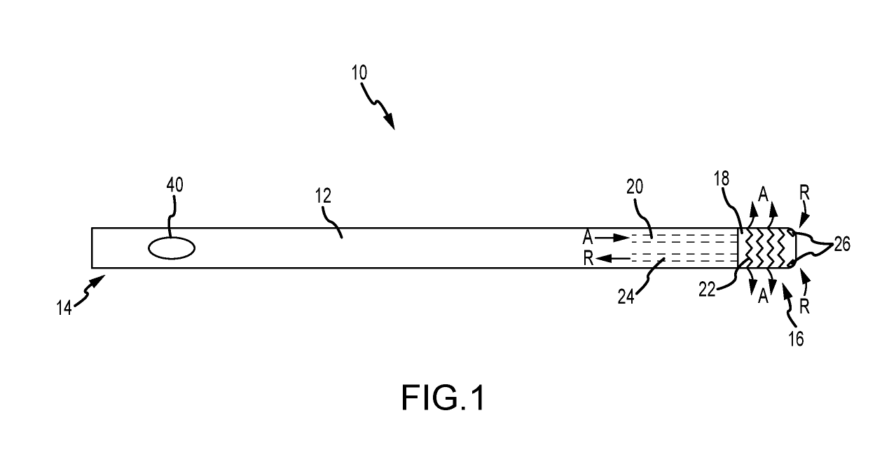

FIG. 1 is a schematic representation of an exemplary irrigated RF ablation catheter 10. Catheter 10 generally includes an elongate body 12 having a proximal portion 14 and a distal portion 16. Distal portion 16 includes a tip electrode 18; certain exemplary configurations of tip electrode 18 are disclosed in U.S. Pat. No. 8,480,669, which is hereby incorporated by reference as though fully set forth herein.

Catheter 10 also includes an irrigation lumen 20, a portion of which is shown in phantom in FIG. 1. It should be understood that irrigation lumen 20 extends along the length of catheter 10 to proximal portion 14 thereof, where it can be coupled to a pump and irrigant (e.g., saline) source, as described below. Although only a single irrigation lumen 20 is depicted in FIG. 1, additional irrigation lumens 20 can be provided without departing from the scope of the present disclosure.

Irrigation lumen 20 distally terminates at one or more irrigation orifices within distal portion 16 of catheter 10. These irrigation orifices extend through the wall of catheter 10 to allow irrigant to be exhausted from catheter 10 in vivo. As shown in FIG. 1, the irrigation orifices are in the form of gaps 22 in tip electrode 18 (see U.S. Pat. No. 8,480,669). The flowpath of irrigant through irrigation lumen 20 and out of gaps 22 in tip electrode 18 is represented by arrows “A” (antegrade flow) in FIG. 1.

Catheter 10 further includes at least one drainage lumen 24, a portion of which is shown in phantom in FIG. 1. As with irrigation lumen 20, drainage lumen 24 extends along the length of catheter 10 to proximal portion 14 thereof, where it can be coupled to a pump and drain, as described below. Although only a single drainage lumen 24 is depicted in FIG. 1, additional drainage lumens 24 can be provided without departing from the scope of the present disclosure.

Drainage lumen 24 distally terminates at one or more drainage orifices 26 within distal portion 16 of catheter 10. Drainage orifices 26 extend through the wall of catheter 10 to allow fluid to be drawn into catheter 10 for exhaustion outside the body. The flowpath of fluid into drainage orifices 26 and through drainage lumen 24 is represented by arrows “R” (retrograde flow) in FIG. 1.

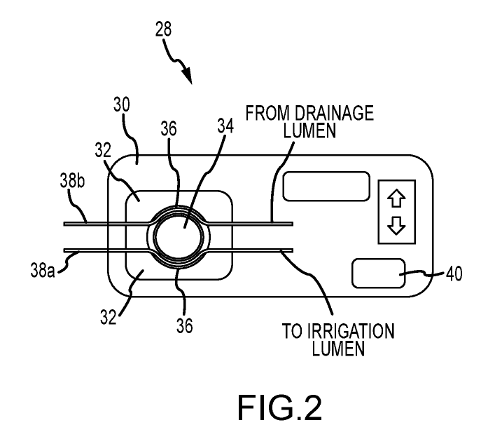

FIG. 2 depicts a pump 28. As depicted in FIG. 2, pump 28 is a peristaltic pump, such as the Cool Point™ irrigation pump of Abbott Laboratories. The configuration and operation of pump 28 will be familiar to those of ordinary skill in the art (see, e.g., United States patent application publication no. 2007/0224063, which is hereby incorporated by reference as though fully set forth herein).

The ordinarily skilled artisan will appreciate that pump 28 generally includes a housing 30, one or more clamps 32, and a rotor 34. Rotor 34 includes a plurality of roller spaced about its circumference and is mounted to rotate about an axle.

Tubing channels 36 are defined between clamps 32 and rotor 34. Tubing channels 36 accommodate tubing 38a and 38b. The portion of tubing 38a, 38b positioned between clamps 32 and rotor 34 is referred to herein as the “interface tubing.” One of ordinary skill in the art will appreciate that, as rotor 34 turns, the rollers will periodically (if evenly spaced about the circumference of rotor 34) impinge upon the interface tubing, pushing tubing 38a, 38b against clamps 32 and forcing fluid through tubing 38a, 38b to provide a pulsatile flow therethrough.

One end of tubing 38a can be coupled to a suitable reservoir of irrigation fluid, such as saline, while the opposite end of tubing 38a can be coupled to catheter 10, and more particularly to irrigation lumen 20. Thus, when in operation, pump 28 moves irrigation fluid from the reservoir into catheter 10, where it moves through irrigation lumen 20 and exits via gaps 22 in tip electrode 18.

Similarly, one end of tubing 38b can be coupled to a drain, while the opposite end of tubing 38b can be coupled to catheter 10, and more particularly to drainage lumen 24. Thus, when in operation, pump 28 draws fluid in through drainage orifices 26, along drainage lumen 24, and exhausts it into a drain.

In the configuration shown in FIG. 2, fluid will be extracted simultaneously with the delivery of irrigant. The rate at which fluid is extracted will also be equal to the rate at which irrigant is delivered. This is referred to herein as “synchronized” aspiration.

In other embodiments, the rate at which fluid is extracted can be independent of the rate at which irrigant is delivered (referred to herein as “manual” aspiration). For example, irrigation lumen 20 and drainage lumen 24 can be connected to separate, independently operable pumps 28 or to separate, independently operable rotors 34 on a single pump 28.

Various manual aspiration modes are contemplated. In a “constant” manual aspiration mode, the rate at which fluid is extracted is constant.

In a “controlled” manual aspiration mode, the rate at which fluid is extracted can be responsive to one or more monitored parameters. For example, a feedback controller, which can be incorporated into pump 28, can monitor one or more parameters such as impedance variation, vascular pressure, and/or RF delivery time vs. volume pumped. The feedback controller can compare the monitored parameter(s) to respective threshold(s), activating aspiration when the monitored parameter(s) cross the threshold(s) in one direction and deactivating aspiration when the monitored parameter(s) cross the threshold(s) in the opposite direction.

For example, the feedback controller can activate aspiration when two or more of the following criteria are met: (1) vascular pressure increases over an initial vascular pressure by about 10%; (2) impedance fluctuates by more than about 10% of a baseline (e.g., pre-irrigation) level; and (3) pumped irrigant volume of about 50 mL.

Likewise, the feedback controller can deactivate aspiration when the retrograde flow volume reaches about 90% of the pumped irrigant volume.

A suitable control switch (e.g., slider switch 40) can be provided on catheter 10 and/or pump 28 to toggle between synchronized aspiration, constant manual aspiration, controlled manual aspiration, and/or aspiration off modes.

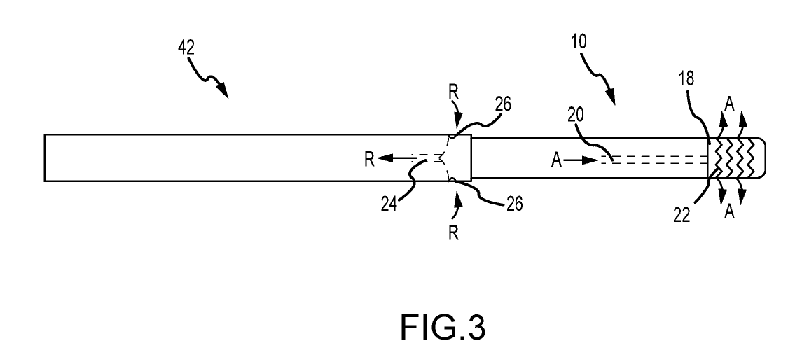

Another embodiment is depicted in FIG. 3. As shown in FIG. 3, catheter 10 is advanced through a sheath 42. Further, drainage lumen 24 and drainage orifices 26 are provided on sheath 42, rather than on catheter 10.

In use, for example to perform epicardial ablation, catheter 10 is introduced into a pericardial space, for example via sheath 42, using techniques that will be familiar to those of ordinary skill in the art. Irrigation lumen 20 is then coupled to pump 28 (e.g., to tubing 38a) to deliver irrigant through irrigation orifices 22. Likewise, drainage lumen 24 is coupled to pump 28 (e.g., to tubing 38b) to drain fluid from the pericardial space.

The practitioner can then select an aspiration mode (e.g., off, synchronized, constant manual, controlled manual) using switch 40.

Tip electrode 18 can then be activated, for example via an RF generator, to deliver RF energy to ablate the tissue while simultaneously delivering irrigant through irrigation orifices 22 and draining fluid via drainage lumen 24 according to the selected aspiration mode.

Although several embodiments of this invention have been described above with a certain degree of particularity, those skilled in the art could make numerous alterations to the disclosed embodiments without departing from the spirit or scope of this invention.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. An irrigated ablation system comprising:
at least one medical device comprising:
at least one irrigation lumen extending along a length of the at least one medical device and having a distal terminus at an irrigation orifice that extends through a wall of a distal portion of the at least one medical device; and
at least one drainage lumen extending along the length of the at least one medical device and having a distal terminus at a drainage orifice that extends through the wall of the distal portion of the at least one medical device; and
at least one pump coupled to the at least one irrigation lumen and to the at least one drainage lumen,
wherein the at least one pump is operable to deliver irrigant through the at least one irrigation lumen and to extract fluid through the at least one drainage lumen.

2. The irrigated ablation system according to claim 1, wherein the at least one medical device comprises:
an ablation catheter; and
a sheath dimensioned to receive the ablation catheter therethrough, and
wherein the ablation catheter comprises the at least one irrigation lumen and the irrigation orifice, and
wherein the sheath comprises the at least one drainage lumen and the drainage orifice.

3. The irrigated ablation system according to claim 1, wherein a rate at which the at least one pump extracts fluid through the at least one drainage lumen equals a rate at which the at least one pump delivers irrigant through the at least one irrigation lumen.

4. The irrigated ablation system according to claim 3, wherein the at least one pump comprises a peristaltic pump simultaneously operable to deliver irrigant through the at least one irrigation lumen and to extract fluid through the at least one drainage lumen.

5. The irrigated ablation system according to claim 4, wherein the peristaltic pump comprises a rotor, and wherein an interface tubing for the at least one irrigation lumen and an interface tubing for the at least one drainage lumen are positioned on opposing sides of the rotor.

6. The irrigated ablation system according to claim 1, wherein a rate at which the at least one pump extracts fluid through the at least one drainage lumen is independent of a rate at which the at least one pump delivers irrigant through the at least one irrigation lumen.

7. The irrigated ablation system according to claim 6, wherein the rate at which the at least one pump extracts fluid through the at least one drainage lumen is constant.

8. The irrigated ablation system according to claim 6, further comprising a controller operable to control the rate at which the at least one pump extracts fluid through the at least one drainage lumen responsive to one or more monitored parameters.

9. The irrigated ablation system according to claim 8, wherein the one or more monitored parameters are selected from the group consisting of impedance; pressure; ablation time; and irrigant volume.

10. A method of performing cardiac ablation comprising:
introducing at least one medical device into a pericardial space, the at least one medical device comprising:
at least one irrigation lumen extending along a length of the at least one medical device and having a distal terminus at an irrigation orifice that extends through a wall of a distal portion of the at least one medical device and that is open to the pericardial space; and
at least one drainage lumen extending along the length of the at least one medical device and having a distal terminus at a drainage orifice that extends through the wall of the distal portion of the at least one medical device and that is open to the pericardial space;
coupling at least one pump to the at least one irrigation lumen and the at least one drainage lumen such that the at least one pump is operable to deliver irrigant through the at least one irrigation lumen and to extract fluid through the at least one drainage lumen; and
ablating cardiac tissue using the at least one medical device while delivering irrigant through the at least one irrigation lumen and extracting fluid through the at least one drainage lumen.

11. The method according to claim 10, wherein introducing at least one medical device into a pericardial space comprises introducing an ablation catheter into the pericardial space via a sheath, wherein the ablation catheter comprises the at least one irrigation lumen and the irrigation orifice, and wherein the sheath comprises the at least one drainage lumen and the drainage orifice.

12. The method according to claim 10, wherein a rate at which the at least one pump extracts fluid through the at least one drainage lumen equals a rate at which the at least one pump delivers irrigant through the at least one irrigation lumen.

13. The method according to claim 12, wherein coupling at least one pump to the at least one irrigation lumen and the at least one drainage lumen such that the at least one pump is operable to deliver irrigant through the at least one irrigation lumen and to extract fluid through the at least one drainage lumen comprises coupling a peristaltic pump to the at least one irrigation lumen and the at least one drainage lumen such that the peristaltic pump is simultaneously operable to deliver irrigant through the at least one irrigation lumen and to extract fluid through the at least one drainage lumen.

14. The method according to claim 13, wherein coupling a peristaltic pump to the at least one irrigation lumen and the at least one drainage lumen such that the peristaltic pump is simultaneously operable to deliver irrigant through the at least one irrigation lumen and to extract fluid through the at least one drainage lumen comprises:
positioning an interface tubing for the at least one irrigation lumen on a first side of a pump rotor; and
positioning an interface tubing for the at least one drainage lumen on a second side of the pump rotor opposite the first side of the pump rotor.

15. The method according to claim 10, wherein a rate at which the at least one pump extracts fluid through the at least one drainage lumen is independent of a rate at which the at least one pump delivers irrigant through the at least one irrigation lumen.

16. The method according to claim 15, wherein the rate at which the at least one pump extracts fluid through the at least one drainage lumen is constant.

17. The method according to claim 15, further comprising:
monitoring one or more parameters during the ablating step; and
adjusting the rate at which the at least one pump extracts fluid through the at least one drainage lumen responsive to the one or more monitored parameters.

18. The method according to claim 17, wherein the one or more monitored parameters are selected from the group consisting of impedance; pressure; ablation time; and irrigant volume.

19. An irrigated ablation system comprising:
a medical device comprising:
an irrigation lumen extending along a length of the medical device and having a distal terminus at an irrigation orifice that extends through a wall of a distal portion of the medical device; and
a drainage lumen extending along the length of the medical device and having a distal terminus at a drainage orifice that extends through the wall of the distal portion of the medical device;
at least one pump coupled to the irrigation lumen and to the at least one drainage lumen, wherein the at least one pump is operable to deliver irrigant through the irrigation lumen and to extract fluid through the drainage lumen; and
a control to toggle the at least one pump between a first operating mode, wherein a rate at which the at least one pump extracts fluid through the drainage lumen equals a rate at which the at least one pump delivers irrigant through the irrigation lumen, and a second operating mode, wherein the rate at which the at least one pump extracts fluid through the drainage lumen is independent of the rate at which the at least one pump delivers irrigant through the irrigation lumen.

20. The irrigated ablation system according to claim 19, wherein the second operating mode includes a first sub-mode, wherein the rate at which the at least one pump extracts fluid through the drainage lumen is constant, and a second sub-mode, wherein the rate at which the at least one pump extracts fluid through the drainage lumen is responsive to one or more monitored parameters selected from the group consisting of impedance; pressure; ablation time; and irrigant volume.
